(19) United States
(12) Patent Application Publication
(10) Pub. No.: US 2019/0314191 A1
(43) Pub. Date: Oct. 17, 2019

(54) MermaidCup

(71) Applicant: WENDY BOBARIKIN, TULSA, OK (US)

(72) Inventor: WENDY BOBARIKIN, TULSA, OK (US)

(21) Appl. No.: 15/898,609

(22) Filed: Apr. 13, 2018

(51) Int. Cl. A61F 5/455

(52) U.S. Cl. CPC A61F 5/4553

(57) ABSTRACT

A menstrual container, generally a cup configuration, incorporating a top with airholes to help break the suction when pulling out with the knob. A cylindrical elongated body with fish scale texture for better prehending, two rims designed around the top of the body to fortify anti leak properties and a knob at the bottom for facile pulling out of the cup.

### FIELD OF INVENTION

The present invention relates to obstetrical-gynecological devices and more specifically to menstrual cup having decorative fish scales to provide for better grip and anti-leakage properties with a double rim design around the body. The invention, substitutable for conventional menstrual cups, which will provide further convenience, better aesthetics and satisfaction for the user without compromising the functionality of typical menstrual containers.

### BACKGROUND

Menstrual cups have actually been around since the 1930s, but America was slow to catch on. The first menstrual cup for U.S. use was manufactured in 1987. Since then, there have been several others produced, manufactured from different substances ranging from rubber to silicone.

A menstrual cup is a feminine hygiene product that is inserted into the vagina during menstruation. Its purpose is to prevent menstrual fluid (blood containing uterine lining) from leaking onto clothes. Menstrual cups are usually made of flexible medical grade silicone and shaped like a bell with a stem. The stem is used for insertion and removal. The bell-shaped cup seals against the vaginal wall just below the cervix. Every 4-12 hours (depending on the amount of flow), the cup is removed, emptied, rinsed, and reinserted.

Unlike tampons and pads, cups collect menstrual fluid rather than absorbing it. One cup is reusable for up to five years or more. This makes their long-term cost lower than that of disposable tampons or pads, though the initial cost is higher. Menstrual cups are also known for their practical use and are more eco-friendly than pads or tampons.

According to U.S. Pat. No. 3,404,682A issued on 1965 Nov. 17 to Barbara M Waldron a vaginal cup having a lower sealing ring in addition to those disposed at the upper end thereof, as an aid to preventing involuntary expulsion or shifting of the cup after proper placement. It is also having improved method for removal after use. However removal of Waldron's cup is even more difficult and uncomfortable for the user than the insertion. Furthermore on its removal, spillage of blood is a very frequent occurrence.

In menstrual cups known in the prior art, as exemplified, for example, in the Chalmers U.S. Pat. No. 2,089,113, of Aug. 3, 1937, the device includes a relatively smooth outer surface with a rim and projecting ring intended to create a seal with the walls of the vaginal canal. This type of seal, although not without utility, has proven ineffectual in many cases, due to improper physiological fit caused by the irregular convolutions or folds in the vaginal mucosa and wide individual differences to which a single commercially-produced size cannot be adapted. A poor seal, permitting leakage of menstrual fluid, negates the purpose of the entire product. It is therefore among the principal objects of the present invention to provide a menstrual cup of the class described having an improved seal which provides greatly improved sealing action without causing any physical discomfort.

It is also known in the art, as exemplified in the Chalmers U.S. Pat. No. 2,534,900 of Dec. 19, 1950, to provide a tapered cup which incorporates a thickened wall portion along the point to be placed posteriorly, and which terminates in a bulge or keel-like appendage. This construction has not only proven impractical from a positioning standpoint, as it is very difficult to fold and insert, but also has been found to be extremely uncomfortable due to the hard mass of the thickened wall portion which tends to exert pressure on the colon, especially when full.

According to U.S. Pat. No. 5,947,992A of issued on 1995 Feb. 21 to Filiberto P. Zadini and Giorgio C. Zadini is an intravaginal inflatable member impermeable to fluids adapted to collect upon inflation, menstrual blood and also capable of provide sealable closure of the vaginal canal for the prevention of vaginal exit of menstrual blood. By being inflatable, the device is easily inserted and removed when deflated and is also capable of sealing off the blood collected avoiding the spillage of blood upon extraction and disposal. The inflation of the device can be conveniently initiated prior to insertion into vagina and completed with the device fully inserted or can be initiated and completed with the device already inserted into the vagina. Inflation of the device can be achieved without the use of an external pneumatic source, but with a self-inflating apparatus within the device. The inflatable member can be used as a standing alone device or in combination with a vaginal tampon for the prevention of blood leakage.

Regardless of the individual differences among the menstrual blood collector cups, the general design of all these devices makes their insertion into the vaginal canal an uncomfortable and difficult task. Moreover, the manufacturing material isn't safe as well to use for long term.

None of the previous inventions and patents, taken either singly or in combination, is seen to describe the instant invention as claimed with the non-slip features because of fish scales and anti-leak properties because of double rims present across the body. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary object of the invention is related to a menstrual cup which has fish scale texture to help with the grip for removal and sealing.

Further it is the object of the invention to provide double rims around the upper exterior body of cup to avoid blood leakage and maintaining the shape of cup.

Furthermore, it is the object of the invention to provide air holes on the top of cup to break the suction when pulling out.

Another object of the invention lies in the provision of an improved menstrual cup of increased capacity.

The invention will also provide user with aesthetic satisfaction by changing the outlook of conventional cup to mermaid designed menstrual cup.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a side view of the present invention.

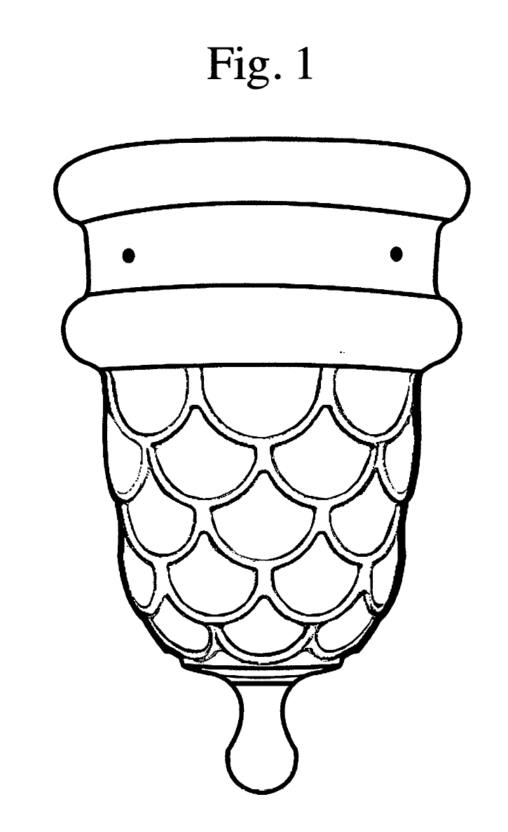

FIG. 2 is a top view of present invention.

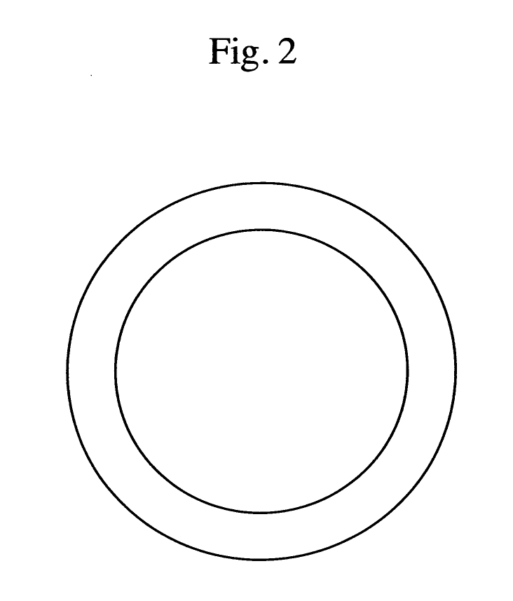

FIG. 3 is showing internal thickness of cup.

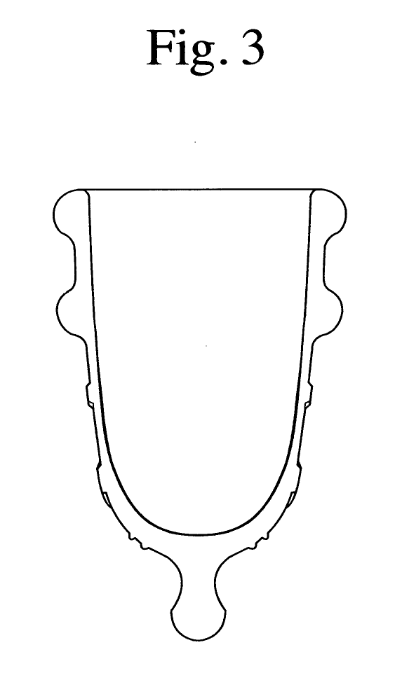

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Menstrual cups are a well-known utility now a day. This is because their advantages over conventional ways. Menstrual cups are ecofriendly as well as wallet friendly. A reusable cup costs not more than $40 and can last up to 10 years. That means less waste in landfills and less money over time.

Moreover, these cups can be left for 12 hours. Traditional ways like pads needs to be changed after every 2 to 4 hours whereas tampons need to be changed every 4 to 8 hours, depending on the flow. Menstrual Cups can stay in longer, so they're good for overnight protection and once you get the hang of inserting it, there's no need to wear a backup pad or liner.

A menstrual cup can hold 1 ounce of liquid, roughly twice the amount of a super-absorbent tampon or pad. The difference can be a comfort on your heavy flow days. According to experts it is safer than traditional ways as offer lower risk from infections, toxic waste and other health issues.

The menstrual cup in its current embodiment is designed as a simple, inexpensive catch-basin for menstrual waste and is designed to be self-supporting. Its body is designed in a mermaid like structure with a wider end at the top.

In one embodiment of the invention the menstrual cup is a structure which has a wider end and closes at a tunnel like narrow end. The cup can be folded easily. The basic material of the cup is thin medical grade silicone which is also moisture resistant material. As the material of the cup is elastic so it can take the temporary changes in its shape and will come back to its originality once released. This is because once the cup is inserted it should expand in its original position to provide its functionality.

The cup has 2 rims on its anterior portion of the body. The function of these rims is to provide the required tension and maintain the shape of the cup when inserted and once released. Furthermore the double rims are designed across the anterior portion of body to provide anti leakage function.

In another embodiment of the invention the fish scales from middle section moving towards the lower end or base of the cup. This fish scale texture is present to give better support to the cup and help it remain in place during the muscular contraction of vaginal canal. Furthermore the fish scale texture helps with the grip for removal and sealing. The fish scale also helps for grip during insertion and when rotating the cup to create a proper seal.

In another embodiment of the invention the cup has a knob design at the lower narrow end. The knob helps with easy access and grip to the cup with fingers during removal.

In further embodiment of the invention are the airholes present at the top anterior portion of the cup which allows the release of the gases which form in the vaginal passage due to certain pathological conditions. These gases cause the pressure on the cup and forced it to dislocate. Because of these three tiny holes it becomes easy for air to pass through the cup and thus releasing pressure and maintain the balanced pressure which is required for the cup stability and easy removal.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A menstrual cup to insert in vagina where:
The cup is a mermaid shaped structure comprising of a wall having an inner wall surface describing a cavity customized for collecting fluid and an outer wall surface, having fish scales across the body and the vessel extending from an open wide top end to a closed thin bottom end, the open top end having a predetermined diameter of 40 mm, the vessel wall comprising an upper rim portion at the top end of the vessel for strengthening the top end and to maintain the cup in a pre-selected position in the vagina, a lower rim to maintain the antileakage property and tension level, and a main portion extending downwards from the transition portion to the closed bottom end of the vessel.

2. A mermaid shaped menstrual cup where the outer surface of the cup consists of 2 rims to maintain the anti-leak property of cup.

3. A mermaid shaped cup according to claim 2, where the outer rims functions as tension so that when folded and inserted in the vagina, the menstrual cup pops open on its own.

4. A mermaid shaped cup according to claim 1, where it contains fish like scales from anterior end to narrowing posterior end.

5. A mermaid shaped cup according to claim 1, where the cup has airholes at the top, to break the air pressure for easy release.

6. A mermaid shaped cup according to claim 1, where there is a knob at the bottom to easily pull out the cup when required.

7. A mermaid shaped cup according to claim 1, where the cup is designed to hold a larger amount of fluid.

8. A mermaid shaped cup according to claim 1, where the cup can be used multiple times, just by sterilizing it.

9. A mermaid shaped cup which has a knob at the posterior narrow side that helps in removal of cup.
